(19) United States
(12) Patent Application Publication
(10) Pub. No.: US 2008/0132770 A1
(43) Pub. Date: Jun. 5, 2008

(54) RESEARCH DATA CLASSIFICATION AND QUALITY CONTROL FOR DATA FROM NON-INVASIVE PHYSIOLOGIC SENSORS

(75) Inventors: Eric J. Ayers, Alliquippa, PA (US); Bernard F. Hete, Kittanning, PA (US); Eric W. Starr, Allison Park, PA (US)

Correspondence Address: BLYNN L. SHIDELER;THE BLK LAW GROUP, 3500 BROKKTREE ROAD, SUITE 200, WEXFORD, PA, 15090, US

(73) Assignee: STARR LIFE SCIENCES CORP., Oakmont, PA (US)

(21) Appl. No.: 11/951,194

(22) Filed: Dec. 5, 2007

Related U.S. Application Data
(60) Provisional application No. 60/868,681, filed on Dec. 5, 2006.
(60) Provisional application No. 60/884,392, filed on Jan. 10, 2007.

(51) Int. Cl. A61B 5/00; A61B 5/08

(52) U.S. Cl. 600/323; 600/484

(57) ABSTRACT

A method and apparatus of obtaining research data that includes a set of physiologic parameters of a subject by utilizing a physiologic sensor which measures a plurality of physiologic parameters of a subject by obtaining signals from the sensor from which the physiologic parameters are calculated and which verifies the calculated parameters with preset criteria, comprising the steps of: Providing a plurality of distinct error classification codes for the calculated parameters, wherein each error classification code is associated with a given set of failed criteria; Recording the calculated parameters of the subject over a session period; Assigning error classification codes to calculated parameters that failed to meet the given criteria; and Recording the assigned error codes in a time integrated manner with the calculated parameters of the subject over a session period. The Error codes may be visibly displayed to the user in a variety of formats.

### RELATED APPLICATIONS

This application claims the benefit of U.S. Provisional patent application Ser. No. 60/868,681 filed Dec. 5, 2006 entitled “Research Data Quality Control Software.”

This application claims the benefit of U.S. Provisional patent application Ser. No. 60/884,392 filed Jan. 10, 2007 entitled “Small Animal Pulse Oximeter User Interface.”

### BACKGROUND OF THE INVENTION

1. Field of the Invention

The present invention relates to quality control of research data, and more particularly to quality control and classification of data from non-invasive physiologic sensors such as pulse oximeters.

2. Background Information

Conclusions that are drawn from medical research require that the physiologic data recorded which forms the basis of the medical research be as free of errors as possible. However, sets of physiologic data often include data points that are bad due to external factors such as movement of the subject or movement of the sensor.

Most medical research starts with animals. Among the animals used in research, teaching, and testing, mice comprise a large majority of all experimental mammals. The remarkable genetic similarity of mice to humans, combined with great convenience, perhaps accounts for mice so often being the experimental model of choice in research. Some have estimated that 30-60 million mice are used for research purposes annually. In 2006, the University of California Center For Animal Alternatives conservatively estimated that that well over 6,000,000 mice were used annually for research purposes in the United States alone. Besides being genetically similar to humans, mice are small and inexpensive to maintain. Rats represent the next largest majority of experimental mammals, with the number of rats used annually for research being estimated as ⅓-¼ the number of mice. As an underscore of the importance of mice in research the 2007 Nobel Medicine Prize was awarded to Mr. Capecii, Mr. Evans, and Mr. Smithies for the development of “Knockout Mice” which have been designated as the test-bed of biomedical research for the 21^{st} century.

Animals, in general, are not compliant while their physiologic measurements are being recorded. This causes portions of the data from some physiologic sensors to be bad. The problem is generally exacerbated with smaller animals as the sensors can become relatively larger relative to the animal and, in general, there is increased difficulty in obtaining measurements from smaller subjects. Consequently this can be of great concern for research on rats and mice, which represents the greatest percentage, by far, of mammalian research.

As a representative example, heart rate is usually obtained via electrodes placed on the subject or via light shined through a portion of the subject's anatomy. If the subject moves, the heart beats are distorted by this movement. If heart rate is being monitored while the subject moves a few of the data points will not be correct. The researcher will often conclude his experiment by averaging the data, but the mean of the data can be skewed by this accumulation of errors caused by these environmental factors. As another example, arterial saturation and pulse rate measured by pulse oximetry are measurements that are well known to be effected by motion of the patient. If the subject moves, even slightly, pulse rate and saturation measurements from today's commercial pulse oximeters are ignored for several beats. In order to fill-in the gaps in the data during times of motion, today's pulse oximeters report the last known values of pulse rate and saturation. The problem is that if the subject keeps moving, the last value is continued to be held and thus changes in pulse rate and saturation are not reflected in the research data.

The physiologic sensors used in research are also used throughout the medical field as well. For example, pulse oximeters that are useful in research applications are also the standard of care for measuring pulse rate and oxygen saturation of a patient. Similarly Blood Pressure sensors, EKG sensors and the like used in research are also used in the medical fields. There can be some physiologic sensors useful in research that may have no analogous medical care-giving counterpart, but in general the research devices are also used in the medical fields. Of course this phenomenon is a result of researcher pulling recourses from what is readily available in the medical field.

In the medical fields, there have been a number of physiologic monitors that incorporate an alarm or the like to indicate to the caregiver when a signal is lost and the medical parameters are not available. These are sometimes called “off patient alarms”, as they are often triggered when a sensor is pulled off of the patient. U.S. Pat. No. 7,024,233 discloses a data confidence indicator comprising a plurality of physiological data and a plurality of signal quality measures derived from a physiological sensor output, and is used to indicate to the caregiver when the measured physiologic parameter does not meet the desired quality level for the signal by generating a low signal quality alert for the user. These caregiver alarms and quality signals do not, however, provide the researcher with the information needed to properly evaluate and utilize (or discard) the obtained data sets or selected portions thereof.

It is an object of the present invention to minimize the drawbacks of the existing technology and to provide a tool for researchers.

### SUMMARY OF THE INVENTION

One non-limiting aspect of the present invention provides a method of obtaining research data that includes a set of physiologic parameters of a subject by utilizing a physiologic sensor which measures a plurality of physiologic parameters of a subject by obtaining signals from the sensor from which the physiologic parameters are calculated and which verifies the calculated parameters with preset criteria, the method further comprising the steps of: providing a plurality of distinct error classification codes for the calculated parameters, wherein each error classification code is associated with a given set of failed criteria; recording the calculated parameters of the subject over a session period; assigning error classification codes to calculated parameters that failed to meet the given criteria; and recording the assigned error codes in a time integrated manner with the calculated parameters of the subject over a session period.

In one non-limiting aspect of the invention the sensor is a pulse oximeter and the invention consists of a method and apparatus for advising the user when the pulse oximeter software is ignoring beats and therefore reporting the pulse rate, saturation, or pulse distention from the last discernible pulse or breath rate from the last discernable breath. The present invention is useful for other data types as well. The invention may communicate to the user while in a real-time monitoring mode through a display of messages to communicate when unreliable signals are detected. If the device is recording the data to a file or other recording system, error codes are stored in the data file on a separate recorded channel so the researcher can review the data set and evaluate whether when the data is reliable or not. The error code listing may include a representation or place holder for when no errors are found (e.g., “0=good signal”), wherein all other error codes indicate which signals contain unreliable data and why. Specifically the distinct error codes may include a textual summary of the error (e.g. “2-Lost Pulse”).

In one non-limiting aspect of the invention the physiologic sensor is a pulse oximeter and the error codes include at least one indicative of the calculated pulse parameter failing to meet the criteria set for the pulse measurements and at least one error code indicative of the calculated oxygen saturation parameter failing to meet the criteria set for the oxygen saturation measurements. The error codes may include at least one indicative of the calculated breath rate parameter failing to meet the criteria set for the breath rate measurements.

One non-limiting aspect of the present invention provides a method of displaying data that includes a set of physiologic parameters of a subject obtained by utilizing a physiologic sensor which measures a plurality of physiologic parameters of a subject by obtaining signals from the sensor from which the physiologic parameters are calculated and which verifies the calculated parameters with preset criteria, the method further comprising the steps of: assigning error classification codes to calculated parameters that failed to meet the given criteria, wherein each error classification code is associated with a given set of failed criteria; displaying the calculated parameters to the user, wherein some of the calculated parameters will be displayed to the user in a different color when the calculated parameter fails to meet preset criteria from the color used to display the calculated parameter when the calculated parameter meet the preset criteria; and displaying the assigned error classification code to the user.

The calculated parameters may be numerically and graphically displayed to the user, wherein some of the calculated parameters will be graphically displayed to the user with a different background color when the calculated parameter fails to meet preset criteria from the background color used to graphically display the calculated parameter when the calculated parameter meet the preset criteria.

In one non-limiting aspect of the present invention the physiologic sensor is a non-invasive sensor, such as a pulse oximeter, for small mammals.

One non-limiting aspect of the present invention provides a set of error codes configured to be assigned to and displayed with calculated parameters of a pulse oximeter the error codes include at least one indicative of the calculated pulse parameter failing to meet the criteria set for the pulse measurements and at least one error code indicative of the calculated oxygen saturation parameter failing to meet the criteria set for the oxygen saturation measurements.

One non-limiting aspect of the present invention provides a small mammal pulse oximetry device with data display having a plurality of data error classification codes indicative of distinct errors in the data of the pulse oximetry device.

These and other advantages of the present invention will be described in the description of the preferred embodiments taken together with the attached figures.

### BRIEF DESCRIPTION OF THE FIGURES

FIGS. 1 and 2 are representative charts listing error codes configured to be assigned to, recorded and displayed with calculated parameters of a pulse oximeter in accordance with one non-limiting embodiments of the present invention;

FIG. 3-8 are representative display screenshots of calculated parameters and the error codes in accordance with one embodiment of the present invention.

### DESCRIPTION OF THE PREFERRED EMBODIMENTS

The present invention is directed to quality control of research data, and more particularly to quality control and classification of data from non-invasive physiologic sensors such as pulse oximeters, particularly a physiologic sensor device for small mammals, such as found in many research applications. This effective solution has been incorporated into a commercial a pulse oximeter, and can be easily added to other devices such as blood pressure monitors, breathing monitors, EKG Monitors and the like. This solution is applicable to most any physiologic data that requires a signal to be analyzed for its frequency, change in frequency, amplitude, change in amplitude, power or change in power.

The present invention has been incorporated into a commercial pulse oximeter for small mammals sold under the Mouse Ox™ brand by Starr Life Sciences. Although the fundamental theory of operation remains the same between pulse oxemetry on humans and smaller mammals, there have been difficulties with developing a commercial pulse oximeter acceptable for small mammals, and only a few have been made available, and most of those have been limited in application to large rats (i.e. heart rates lower than around 350 beats per minute). The Mouse Ox™ brand is the only commercial version of a pulse oximeter that effectively obtains meaning results in both rats and mice (mice have heart rates up to around 800 beats per minute). This is relevant as mice form the largest percentage of research mammals. One source places mice at 49% of the research mammals, while another lists it as 66%. Regardless of the exact percentage, the ability to obtain meaningful data from mice through pulse oximetry can be seen to be particularly important to researchers.

The operation of pulse oximetry, in general, is well known in the art, including criteria for verifying the validity of the results. The details of pulse oximetry are not necessary for the understanding of this invention, however, as background, a pulse monitor is one type of non-invasive physiologic sensor and is also called a photoplethysmograph. It typically incorporates an incandescent lamp or light emitting diode (LED) to trans-illuminate an area of the subject, e.g. an appendage, which contains a sufficient amount of blood. In the photoplethysmographic phenomenon the light from the light source disperses throughout the appendage and a light detector, such as a photodiode, is placed on the opposite side of the appendage to record the received light for transmisive type devices or on the same side of the appendage for reflective type devices. Due to the absorption of light by the appendage's tissues and blood the intensity of light received by the photodiode is less than the intensity of light transmitted by the LED. Of the light that is received, only a small portion (that effected by pulsatile arterial blood), usually only about two percent of the light received, behaves in a pulsatile fashion. The beating heart of the subject, and the breathing of the subject as discussed below, creates part of this pulsatile behavior. The “pulsatile portion light” is the signal of interest and effectively forms the photoplethysmograph. The absorption described above can be conceptualized as AC and DC components. The arterial vessels change in size with the beating of the heart and the breathing of the patient. The change in arterial vessel size causes the path length of light to change from dₘᵢₙ to dₘₐₓ. This change in path length produces the AC signal on the photo-detector, I_{L} to I_{H}. The AC Signal is, therefore, also known as the photo-plethysmograph. The absorption of certain wavelengths of light is also related to oxygen saturation levels of the hemoglobin in the blood transfusing the illuminated tissue. In a similar manner to the pulse monitoring, the variation in the light absorption caused by the change in oxygen saturation of the blood allows for the sensors to provide a direct measurement of arterial oxygen saturation, and when used in this context the devices are known as oximeters. The use of such sensors for both pulse monitoring and oxygenation monitoring is known and in such typical uses the devices are often referred to as pulse oximeters.

Pulse oximeters have been proposed for obtaining additional parameters from the received signals. The Mouse Ox™ device, for example, can now provide breath rate measurements, pulse distension measurements (related generally to the work of the heart or blood flow of the subject) and breath distension measurements (related to the work of breathing of the subject). Current commercial pulse oximeters, other than the Mouse Ox™ brand device, do not have the capability to measure these extraneous parameters.

In such devices the output generally includes a display of the sensed parameter as determined by the sensor device to the user in some format on an associated display device.

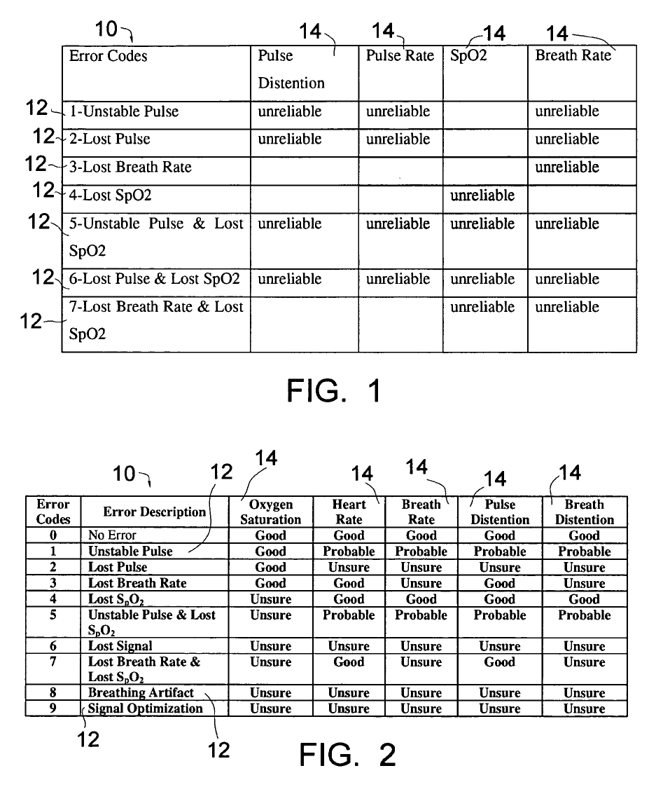

FIGS. 1 and 2 illustrate two charts 10 of representative listings of error codes 12 configured to be assigned to, recorded and displayed with calculated parameters 14 of a pulse oximeter in accordance with one non-limiting embodiments of the present invention. In accordance with one aspect of the present invention each error code 12 includes a short text summary of the associated code. The chart 10 lists some of the associated parameters 14 and can merely identify which ones are unreliable or unsure with a given error code such as in FIG. 1, or the chart 10 may include a listing of which parameters 14 are good (i.e. reliable measurements) and which parameters 14 may be compromised or unsure under the designated code 12. The chart 10 may include other designations, such as “probable” as shown in FIG. 2, for any given chart 10. Specifically in FIG. 2, the designation “Good” means that the given measurement for that parameter 14 satisfies all of the acceptance criteria for the subject device, namely the Mouse Ox™ brand pulse oximeter. The designation “Probable” means that signal quality is beginning to erode but the pertinent displayed values are still valid. The designation “Unsure” means that the given measurement for the parameter 14 does not meet the defined acceptance criteria for that parameter 14. The “unsure” status of the parameter will remain during the presence of that particular code 12. The chart 10 is preferably provided in the user manual and/or available as a help screen to the user on the display so the user can review all of the designated error codes 12. The user may be able to add error codes 12 associated with preset thresholds or alarms that may be selected to be pertinent to the particular project at hand.

The Mouse Ox™ brand pulse oximeter has a series of internal preset acceptance criteria associated with the output parameters 14. The present invention utilizes these present acceptance criteria. Each error code 12 is associated with the failure at least one set of acceptance criteria. It is possible that a plurality of distinct sets of failed acceptance criteria will be associated with one error code 12. In other words there may be two independent acceptance criteria utilized in a device that if either or both are failed then a single associated error code 12 will be triggered. For example, two separate acceptance criteria in the software could be independently indicative of a lost pulse error code 12.

The details of the present invention are not associated with the selection of particular acceptance criteria, as these are generally associated with the device. With pulse oximetry, the error codes 12 can include a lost pulse, a lost breath rate, and a lost oxygenation (SPO2). The lost pulse error code 12 is indicative of the calculated pulse parameter 14 failing to meet the acceptance criteria set for the pulse measurements, and the lost SPO2 error code 12 is indicative of the calculated oxygen saturation parameter 14 failing to meet the criteria set for the oxygen saturation measurements, and the lost breath rate error code 12 is indicative of the calculated breath rate parameter 14 failing to meet the criteria set for the breath rate measurements. Other error codes 12 can be provided for combinations of these error codes, such as Lost Breath Rat and Lost SPO2 codes 12. Further, additional codes 12 can be provided indicative of the failure of other acceptance criteria, such as code 12 indicative of a lost signal, or code 12 indicative of a motion artifact. Effectively any acceptance criteria that can convey meaningful data to a researcher can have a code 12 associated therewith. As a reiteration, the lost pulse error code 12 is indicative of the calculated pulse parameter 14 failing to meet the acceptance criteria set for the pulse measurements, the lost SPO2 error code 12 is indicative of the calculated oxygen saturation parameter 14 failing to meet the acceptance criteria set for the oxygen saturation measurements and the lost breath rate error code 12 is indicative of the calculated breath rate parameter 14 failing to meet the acceptance criteria set for the breath rate measurements.

The chart 10 further includes a “0” error code listing when no errors are present. This is for completeness of the chart 10 and the ability to have data for a complete log file of error codes 12 throughout a session. The “0” error code listing is not technically an error code within the meaning of this specification as it does not comply with the situation of the calculated parameters failing to meet the acceptance criteria and it will not typically be displayed to the user, other than in a review of the log file. The “0” error code is essentially a place holder.

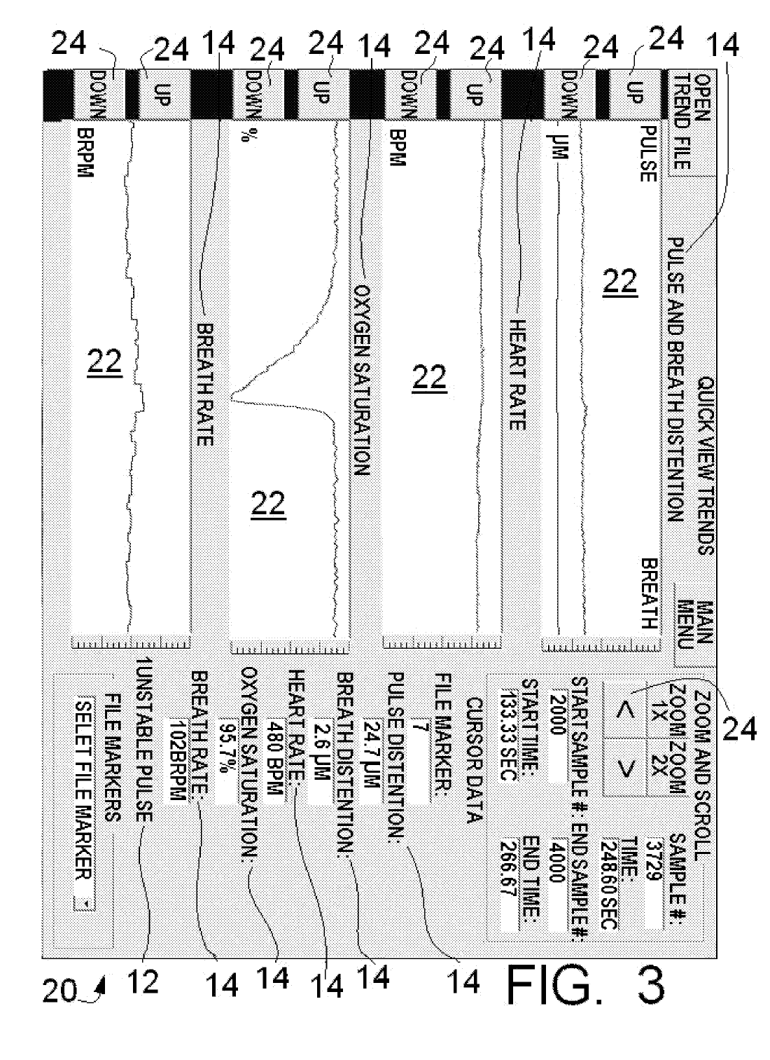

The present invention can be further highlighted in reviewing the possible display screens or screen shots 20 associated with the implementation of the present invention with a Mouse Ox™ brand pulse oximeter. FIG. 3 illustrates a “quick view trends” summary screen 20 of the data collected in the Pulse Oximeter. This screen 20 allows the user to scroll through all the data in an overview fashion with controls 24. The screen 20 of FIG. 3 includes four data charts in the form of graphical displays 22. The four data charts or displays 22 appear on the left side of the monitoring screen 20 and include: (a) Pulse and Breath Distention (micrometers), (b) Heart Rate (bpm), (c) Oxygen Saturation (%), and (d) Breath Rate (brpm). These charts or displays 22 scroll horizontally from right to left, but each data point represents data collected for 1 heartbeat, which means that the data points are not necessarily spaced evenly in time. Each chart 22 in FIG. 3 holds 2000 heartbeats of information across the screen 20, but new data simply continues to scroll. In general, heart-related parameters are displayed in red, while breathing-related parameters are displayed in blue for the easy view of the user. The Pulse and Breath Distention chart 22 includes both pulse and breath distention signals. These signals are included on the same graph because they have similar properties and have the same units. The pulse distention plot is in red, and the breath distention plot is in blue. Descriptors that detail the color coding appear in opposite upper corners of the chart 22.

According to one aspect of the present invention, for all four charts 22, any signal that passes below a user (or present) alarm threshold will cause that chart screen 22 background to change to yellow, and its title to turn red and display a message or error code 12. This visual display of error codes 12 on charts 22 may be limited to only select error codes 12 such as user defined alarms. It is anticipated that the alarm thresholds can be set by the user, and can be considered in addition to the pre-defined acceptance criteria associated with the calculated parameters. For example, the user may set a heart rate alarm threshold with a user defined threshold, and a new error code 12 associated with the failure of the calculated heart rate parameter 14 meeting this user defined threshold. This threshold value may be above the pre-set acceptance criteria for calculating the heart rate for the pulse oximeter (the chart 10 may list the heart rate parameter as being reliable for this error code 12), but may be outside of the parameters needed for the research or otherwise outside of the area of concern. The alarm condition will occur after the first calculated value that falls below the set threshold for each parameter 14. It should be appreciated that some error codes 12 take precedents over others. For example a lost heart rate value error code 12 may be displayed over an error code 12 indicative of the calculated heart rate being below a user defined threshold, and a lost signal error code 12 may take precedence over other error codes 12. It will be obvious to all that the hear rate will be lost if the entire signal is lost.

On the right of the summary screen shot 20 in FIG. 3 are screen controls 24 for moving through the charts, a listing of the starting sample number and ending sample number for the illustrated charts 22, the starting and ending time associated with these data points as well as the specific sample number and associated time that has been selected by the user. The curser may be moved along the charts 22 and click on a selected sample (location on the charts 22) to select it. Alternatively a specific sample number can be typed into the sample number box to be selected. The charts 22 may further include a vertical line indicating the selected sample. There may be a default sample displayed if none is selected, such as the first sample on the charts 22 (left side of charts 22), the last sample on the charts 22 (right side of charts 22) or the mid-point sample (middle of the charts 22).

The data for the selected sample is also illustrated numerically on the right side of the summary screenshot 20 of FIG. 3. Included in this listing, under curser data, is the numerical value for the calculated parameters 14 at the selected sample point. Further the textual summary error code 12 is displayed if this sample is associated with any error codes 12, meaning that if the calculated parameters for that sample fail any of the acceptance criteria. Furthermore, if this error code 12 were associated with any inaccurate or unsure parameter values, then the associated parameters values displayed would be displayed in a visibly different color, such as being grayed out, or possibly omitted altogether. The omission of the displayed value can be easily accomplished by selecting a display color the same or virtually identical to the background color of the numerical display window, such that the “omission” of the displayed value is merely one type of change in color of this displayed value. The value may be omitted altogether, but this complete omission may represent a more complex manipulation of the display software.

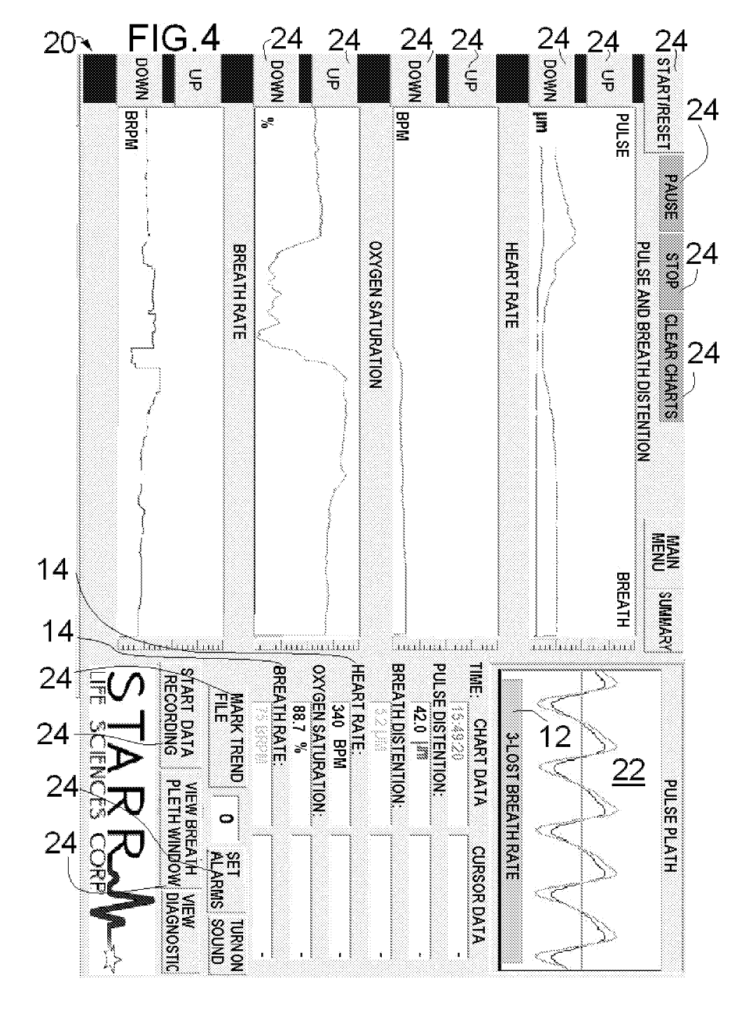

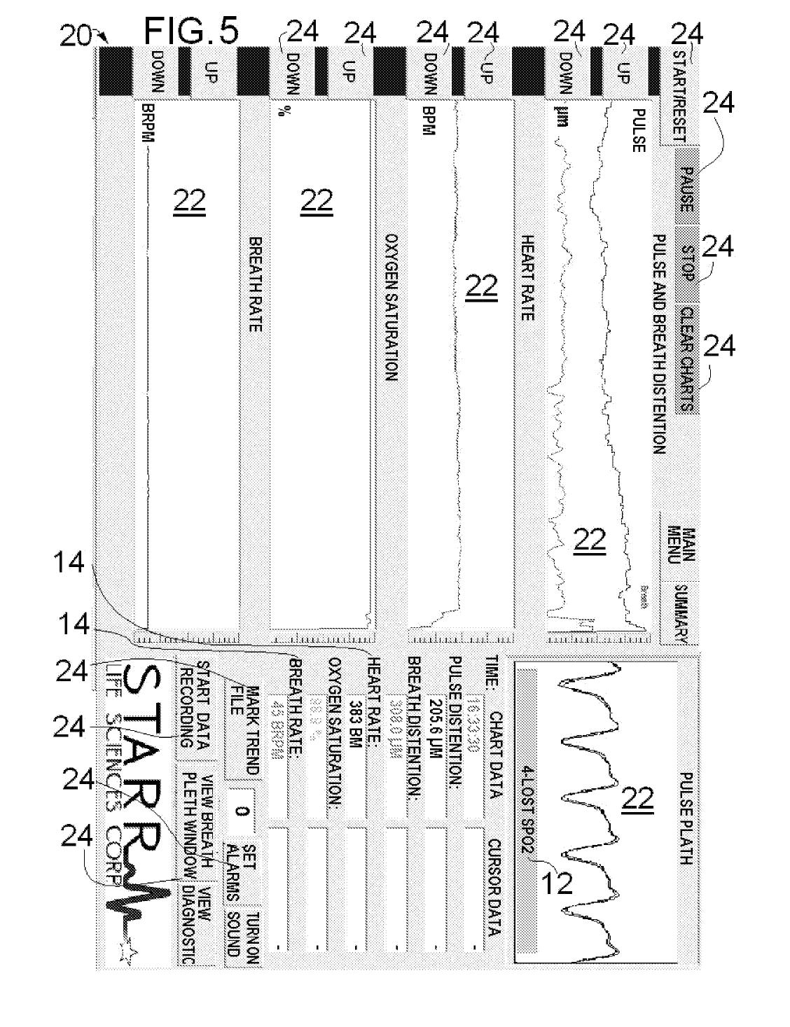

FIG. 4 is a screenshot 20 that is the main data gathering screen for the device. The main screen 20 includes controls 24 for starting and stopping the session, and the data charts described above. The right hand side of the screen includes a data for the most recent sample which will be on the right of the scrolling charts 22 in the real time display, and can include a prior selected sample in the curser data listing. If a prior sample is selected under the curser data, the charts 22 may include a vertical line indicative of this sample (until the selected sample moves off the screen to the left as the samples progress). This screen further includes a Pulse Pleth window or chart 22 that provides a near real-time graphical display of the transmitted red and infrared pulse oximeter light intensities as received by the receiver, to the user. In the manifestation as shown in the figure, the display appears as dual oscilloscope traces. A red trace represents the red transmitted light intensity, while a yellow trace represents the infrared transmitted light intensity. One important utility of this graphical representation of what is effectively raw data is that it allows the user to see the waveforms so that their quality can be judged. Since the quality of the waveforms determines the ability of the pulse oximeter to make continuous accurate measurements of its parameters, displaying them to the user can allow him to be able to move/adjust the sensor location in order to improve signal quality. The raw data traces are sufficient feedback for the user to perceive weaker and stronger signals based upon sensor location (within what ever adjustment is provided in a particular sensor mount). Note that the particular color of the traces is inconsequential, and that the data does not have to be delayed or pre-processed in order to provide beneficial information to the user. Additionally, the processing could be conducted in the same device that has the A/D board and/or the display screen.

Another improvement in data error indication according to the present invention involves letting the user know about problems with the data while the data is being collected through the recording and display of the associated error codes 12. Although the quality of data can be assessed in a general sense using the Pulse Pleth window or chart 22 described above, data signals from the Pulse Pleth window that are judged to be of sufficient quality, may still result in the inability for the software algorithms to successfully calculate one or more parameters at a given instant of time (i.e. they are outside of the acceptance criteria). An additional aid to the user has been provided by changing the color of a given parameter in the data text boxes each time calculation of the associated parameter 14 in the given text box does not pass the acceptance criteria for that parameter 14 that is associated with an error code 12. The error code 12 may be recorded in a log file for such cases that allow the user to flag data that is questionable at a later review. Additionally, a visible indication of a problem is given on the main user screen through a display of the “active” error code 12 with textual summary while data are being collected. This visible error code 12 feedback may, selectively, be done in two additional visible ways. The first is that the background of the Pulse Pleth screen may change color from black to green (and note that the color choices are arbitrary) while a selected error code 12 is active. Secondly, the numerical values displayed in the data text boxes change color when a given parameter 14 does not pass the acceptance criteria for that parameter 14. It has been found beneficial to always utilize the second method of changing the color of the illustrated parameters that are outside the acceptance criteria other than present thresholds, but to change the background of the graphical display of the pulse pleth chart 22 for only selected error codes 12. This differentiation in error code display allows for a grading, of sorts, of error codes 12 for the user. In the illustrated figures the unreliable data is shown in a color matching the background such that it appears to be absent, but the present invention anticipates merely graying out of such questioned data, or use of some other indicative color. Here the color red is used for heart related parameters and blue for breathing related parameters such that other error code indicating colors must, preferably, be selected. This display utility could further include changing the background color on a given data numerical display plot associated with a given error code 12 at a given time.

Turning to the illustrated example of FIG. 4, the error code displayed is a lost breath rate. The breath rate and breath distention measurements are shown grayed out or not visible in the text boxes. Here the background color of the pulse pleth window 22 has not been changed, but the error code 12 is displayed to the user. Turning to the illustrated example of FIG. 5, which is also a main data collection screenshot 20, the error code 12 displayed is a lost SPO2. The SPO2 measurement is shown grayed out or not visible in the numerical display boxes. Here the background color of the pulse pleth window 22 has been changed, and the error code 12 is displayed to the user.

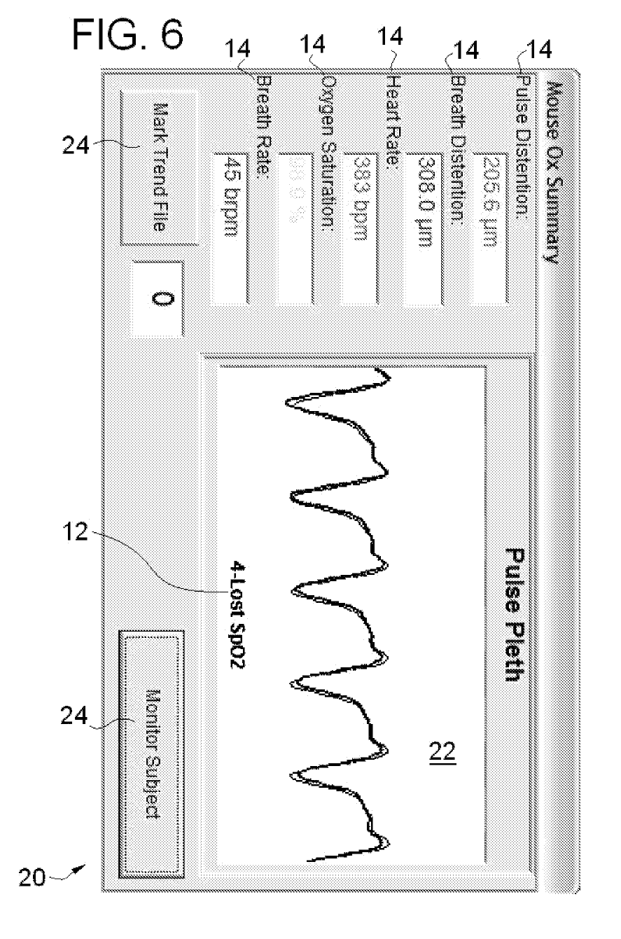

FIG. 6 is another screen shot 20 that is a summary screen of the main data screen shown in FIGS. 4 and 5. This screen can be selected and sized by the user. The error codes 12 are displayed here on chart 22 with the graying out or elimination of the suspect values in the numerical display portion. The background of the graphical chart 22 may or may not be changed in accordance with which error code 12 is active. For the error code 12 shown, namely lost SPO2, the background color of the chart 22 is changed.

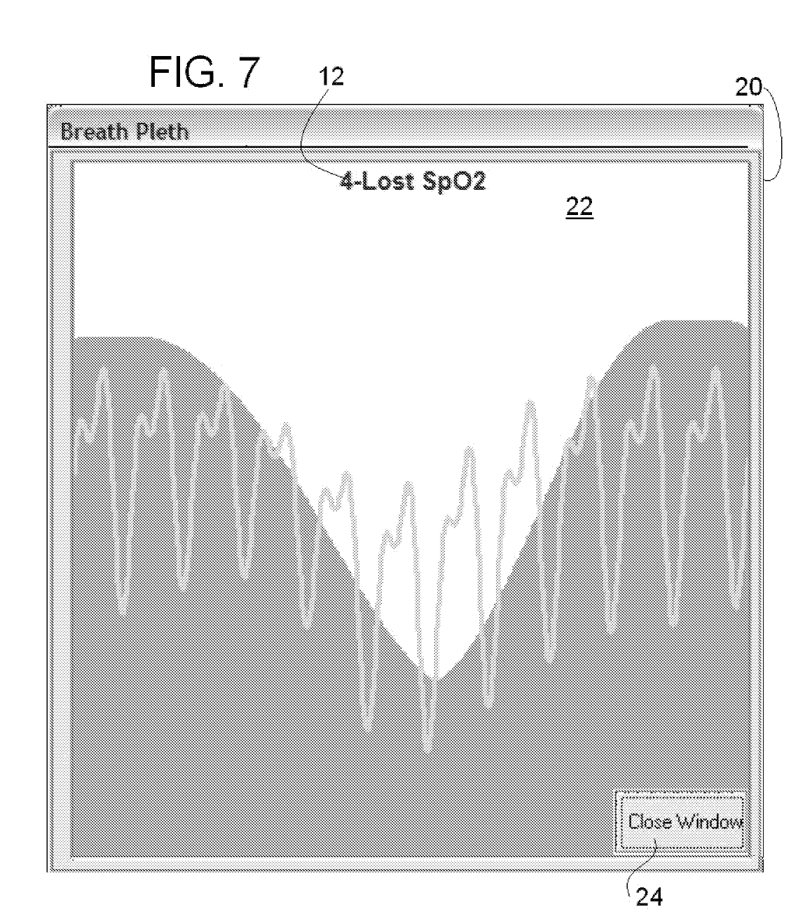

FIG. 7 is another screen shot 20 that is only the Pulse Pleth window that can be individually selected and sized by the user. The error codes 12 are displayed here on chart 22. This window has no numerical display portion for the specific parameters. The background of the graphical chart 22 may or may not be changed in accordance with which error code 12 is active. For the error code 12 shown, namely lost SPO2, the background color of the chart 22 is changed to alert the user.

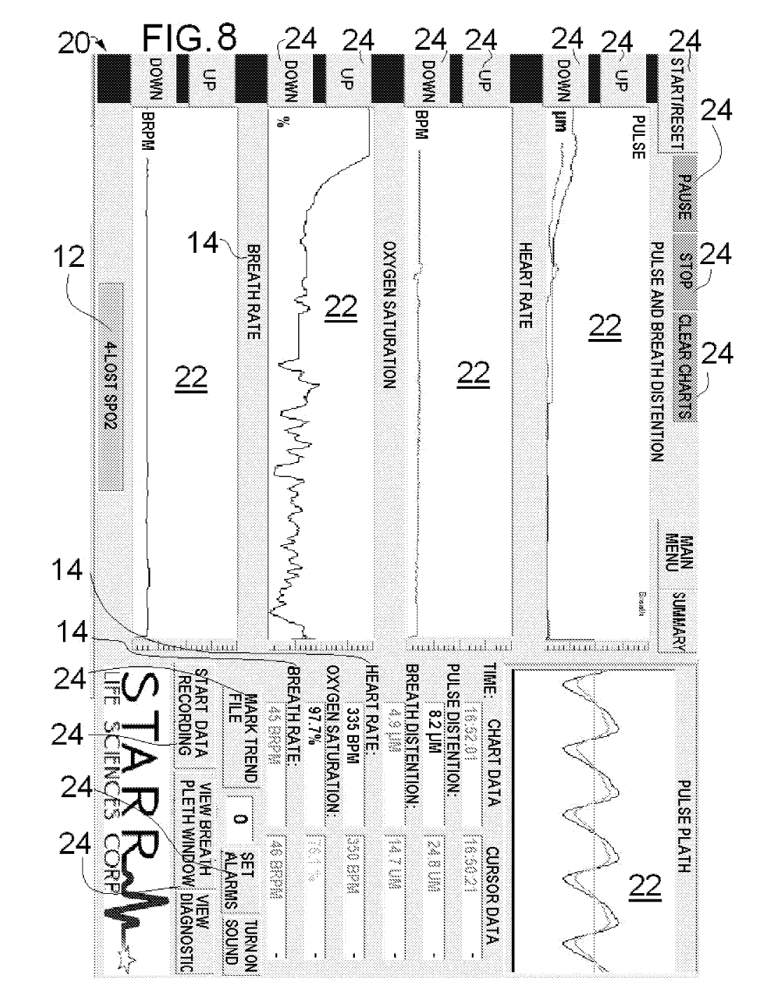

Turning to the illustrated example of FIG. 8, which is also a main data collection screenshot 20, the error code 12 displayed is a lost SPO2. The SPO2 measurement is shown grayed out or not visible in the text boxes. In this example the error code 12 is associated with a prior curser selected sample rather than the current sample. The error code 12 is listed in a location, namely under the left hand charts 22, that can display the selected curser sample number, to indicate that the error code 12 is associated with the earlier sample rather than a contemporaneous measurement. Here the background color of the pulse pleth window 22 has been not been changed for an error code associated with a “historical” or past sample.

Although the present invention has been described with particularity herein, the scope of the present invention is not limited to the specific embodiment disclosed. It will be apparent to those of ordinary skill in the art that various modifications may be made to the present invention without departing from the spirit and scope thereof. The scope of the present invention is defined in the appended claims and equivalents thereto.

What is claimed is:

1. A method of obtaining research data that includes a set of physiologic parameters of a subject by utilizing a physiologic sensor which measures a plurality of physiologic parameters of a subject by obtaining signals from the sensor from which the physiologic parameters are calculated and which verifies the calculated parameters with preset criteria, the method further comprising the steps of:
Providing a plurality of distinct error classification codes for the calculated parameters, wherein each error classification code is associated with a given set of failed criteria;
Recording the calculated parameters of the subject over a session period;
Assigning error classification codes to calculated parameters that failed to meet the given criteria; and
Recording the assigned error codes in a time integrated manner with the calculated parameters of the subject over a session period.

2. The method of obtaining research data according to claim 1 wherein the distinct error codes includes a textual summary of the error.

3. The method of obtaining research data according to claim 2 wherein the calculated parameters and assigned error classification codes are displayed to the user.

4. The method of obtaining research data according to claim 3 wherein some of the calculated parameters will be displayed to the user in a different color when the calculated parameter fails to meet preset criteria from the color used to display the calculated parameter when the calculated parameter meet the preset criteria.

5. The method of obtaining research data according to claim 4 wherein the calculated parameters are numerically and graphically displayed to the user.

6. The method of obtaining research data according to claim 5 wherein some of the calculated parameters will be graphically displayed to the user with a different background color when the calculated parameter fails to meet preset criteria from the background color used to graphically display the calculated parameter when the calculated parameter meet the preset criteria.

7. The method of obtaining research data according to claim 1 wherein physiologic sensor is a non-invasive sensor for small mammals.

8. The method of obtaining research data according to claim 7 wherein physiologic sensor is a pulse oximeter and the error codes include at least one indicative of the calculated pulse parameter failing to meet the criteria set for the pulse measurements and at least one error code indicative of the calculated oxygen saturation parameter failing to meet the criteria set for the oxygen saturation measurements.

9. The method of obtaining research data according to claim 1 wherein physiologic sensor is a pulse oximeter and the error codes include at least one indicative of the calculated pulse parameter failing to meet the criteria set for the pulse measurements and at least one error code indicative of the calculated oxygen saturation parameter failing to meet the criteria set for the oxygen saturation measurements.

10. The method of obtaining research data according to claim 1 wherein physiologic sensor calculates breath rate and the error codes include at least one indicative of the calculated breath rate parameter failing to meet the criteria set for the breath rate measurements.

11. A method of displaying data that includes a set of physiologic parameters of a subject obtained by utilizing a physiologic sensor which measures a plurality of physiologic parameters of a subject by obtaining signals from the sensor from which the physiologic parameters are calculated and which verifies the calculated parameters with preset criteria, the method further comprising the steps of:
Assigning error classification codes to calculated parameters that failed to meet the given criteria, wherein each error classification code is associated with a given set of failed criteria;
Displaying the calculated parameters to the user, wherein some of the calculated parameters will be displayed to the user in a different color when the calculated parameter fails to meet preset criteria from the color used to display the calculated parameter when the calculated parameter meet the preset criteria; and
Displaying the assigned error classification code to the user.

12. The method of displaying data according to claim 11 wherein the error codes includes a textual summary of the error.

13. The method of displaying data according to claim 11 wherein the calculated parameters are numerically and graphically displayed to the user.

14. The method of displaying data according to claim 13 wherein some of the calculated parameters will be graphically displayed to the user with a different background color when the calculated parameter fails to meet preset criteria from the background color used to graphically display the calculated parameter when the calculated parameter meet the preset criteria.

15. The method of displaying data according to claim 11 wherein physiologic sensor is a non-invasive sensor for small mammals.

16. The method of displaying data according to claim 15 wherein physiologic sensor is a pulse oximeter and the error codes include at least one indicative of the calculated pulse parameter failing to meet the criteria set for the pulse measurements and at least one error code indicative of the calculated oxygen saturation parameter failing to meet the criteria set for the oxygen saturation measurements.

17. The method of displaying data according to claim 11 wherein physiologic sensor is a pulse oximeter and the error codes include at least one indicative of the calculated pulse parameter failing to meet the criteria set for the pulse measurements and at least one error code indicative of the calculated oxygen saturation parameter failing to meet the criteria set for the oxygen saturation measurements.

18. The method of displaying data according to claim 11 wherein physiologic sensor calculates breath rate and the error codes include at least one indicative of the calculated breath rate parameter failing to meet the criteria set for the breath rate measurements.

19. A set of error codes configured to be assigned to and displayed with calculated parameters of a pulse oximeter the error codes include at least one indicative of the calculated pulse parameter failing to meet the criteria set for the pulse measurements and at least one error code indicative of the calculated oxygen saturation parameter failing to meet the criteria set for the oxygen saturation measurements.

20. A small mammal pulse oximetry device with data display having a plurality of data error classification codes indicative of distinct errors in the data of the pulse oximetry device.
